(19) United States
(12) Patent Application Publication
(10) Pub. No.: US 2007/0158227 A1
(43) Pub. Date: Jul. 12, 2007

(54) Plaster enclosing packaging bag

(76) Inventors: Satoshi Amano, Ibaraki (JP); Sachiko Honma, Ibaraki (JP); Tetsuro Tateishi, Ibaraki (JP); Naruhito Higo, Saga (JP)

Correspondence Address: NATH & ASSOCIATES, 112 South West Street, Alexandria, VA, 22314, US

(21) Appl. No.: 10/587,834

(22) PCT Filed: Jan. 28, 2005
(86) PCT No.: PCT/JP05/01272

§ 371 Date: Sep. 25, 2006

(30) Foreign Application Priority Data
Jan. 30, 2004 (JP) 2004-024446

(51) Int. Cl. A61B 19/02; B65D 73/00; B65D 81/26

(52) U.S. Cl. 206/438; 206/484; 206/204

(57) ABSTRACT

An object of the present invention is to provide a patch-containing packaging pouch which enables stable storage of a patch containing bisoprolol in a pressure-sensitive adhesive layer. A packaged desiccant 1 of the present invention has a packaging pouch 8 composed of a pair of laminated package members 7a and 7b arranged in mutual opposition, and a patch 10 housed in the space within this packaging pouch 8. Patch 10 is provided with a support 12 and a pressure-sensitive adhesive layer 14 laminated substantially over the entire surface of one side of this support 12. In addition, the pressure-sensitive adhesive layer is composed of a pressure-sensitive adhesive composition containing a pressure-sensitive adhesive and bisoprolol or pharmaceutically acceptable salt thereof. Moreover, a packaged desiccant 20 is housed within packaging pouch 8. The relative humidity within packaging pouch 8 is maintained at 25% or less in a patch-containing packaging pouch 1 having this constitution.

### TECHNICAL FIELD

The present invention relates to a patch-containing packaging pouch, and more particularly, to a patch-containing packaging pouch having a packaging pouch and a patch housed therein.

### BACKGROUND ART

Bisoprolol (and particularly bisoprolol hemifumarate) is widely known as a drug that acts to selectively block β₁ receptors of sympathetic nerves, and is used in the treatment of such conditions as hypertension, angina pectoris and arrhythmia (tachycardia).

This bisoprolol is typically formed into tablets and administered orally. However, since bisoprolol has the property of being extremely susceptible to hydrolysis reactions, in the case of being allowed to stand in air, the bisoprolol in the tablet frequently decreases over time. In other words, tablets containing bisoprolol had low storage stability.

Therefore, various attempts were made to improve the storage stability of tablets containing bisoprolol. For example, tablets are known that are composed of a composition containing bisoprolol, lactic acid and specific cellulose derivative (see, for example, Japanese Patent Application Laid-open No. 2002-308762). In tablets having this composition, long-term storage of the tablets is possible since there is little occurrence of hydrolysis of the bisoprolol.

### DISCLOSURE OF THE INVENTION

In recent years, however, transdermal administration using a patch has attracted attention as a method for administering a drug instead of oral administration. Widely known examples of such patches include those having a constitution in which a pressure-sensitive adhesive layer containing a drug is formed on the surface of support such as a film composed of a resin material or woven fabric. These patches are used by being adhered to the body such that the pressure-sensitive adhesive layer contacts the skin surface. The effects of the drug on the body are then demonstrated as a result of the drug present in the pressure-sensitive adhesive layer passing through the skin and penetrating into the blood.

In addition to enabling a drug to be administered continuously over a long period of time, transdermal administration using a patch also offers the advantage of being able to strictly control the dosage. Consequently, transdermal administration using a patch makes it possible to inhibit an excess amount of drug from being incorporated into the body, thereby leading to expectations of being able to reduce adverse side effects caused by drugs.

Under such circumstances, studies have also begun to apply the above-mentioned bisoprolol to a patch in the same manner as other drugs. However, the bisoprolol introduced into the pressure-sensitive adhesive layer of the patch was more susceptible to hydrolysis reactions as compared with that formed into tablets as in the previously described prior art. Consequently, sufficiently practical long-term storage of patches containing bisoprolol in the pressure-sensitive adhesive layer tended to be difficult.

With the foregoing in view, it is an object of the present invention to provide a patch-containing packaging pouch which enables stable storage of a patch containing bisoprolol in a pressure-sensitive adhesive layer.

The inventors of the present invention conducted various studies to achieve long-term storage of a patch containing bisoprolol in a pressure-sensitive adhesive layer. First, storage was attempted with the patch housed in a packaging pouch, namely in the form of a patch-containing packaging pouch, to prevent contact between the patch and air. However, results were obtained in which inhibition of hydrolysis of the bisoprolol in a pressure-sensitive adhesive layer was difficult even if contact with the outside air was prevented by the package. On the basis of this finding, it was determined that it is difficult to storage such a patch for a long-period of time simply by housing the patch in a packaging pouch.

Next, tests were conducted in which the patch was stored under various conditions to study in detail the hydrolysis reaction of bisoprolol. As a result, in the case of storing the patch under conditions such that the relative humidity exceeded 25% at 25° C., the hydrolysis reaction of the bisoprolol in the pressure-sensitive adhesive layer was found to proceed prominently. Therefore, a study of the relative humidity within the packaging pouch in the above-mentioned patch-containing packaging pouch confirmed that relative humidity had exceeded the above-mentioned value in all cases.

On the basis of these findings, the present invention is a patch-containing packaging pouch comprising: a packaging pouch, and a patch, housed within this packaging pouch, in which a pressure-sensitive adhesive layer is formed on one side of a support; wherein, the pressure-sensitive adhesive layer is formed of a pressure-sensitive adhesive composition containing a pressure-sensitive adhesive and bisoprolol or a pharmaceutically acceptable salt thereof, and the relative humidity inside the packaging pouch at 25° C. is maintained at 25% or less.

Here, relative humidity at 25° C. refers to the ratio (%) of the amount of water vapor (kg·m⁻³) actually contained in the packaging pouch based on a value of 100 for the maximum amount of water vapor (kg·m⁻³: saturation humidity) which can be retained by air within a fixed volume at 25° C.

In this manner, in a patch-containing packaging pouch having the above-mentioned constitution, the patch is stored within a packaging pouch in which the relative humidity therein is maintained so as to be 25% or less at 25° C. As previously described, there is extremely little hydrolysis of bisoprolol in the pressure-sensitive adhesive layer under such conditions. Consequently, according to the patch-containing packaging pouch of the present invention, the reduction in the amount of bisoprolol in the pressure-sensitive adhesive layer of the patch over time is inhibited. As a result, a patch containing bisoprolol in a pressure-sensitive adhesive layer can be easily stored for a long period of time.

The inside of the packaging pouch of the patch-containing packaging pouch of the present invention is preferably such that the above-mentioned relative humidity at 25° C. is maintained at 10% or less. Under these conditions, the hydrolysis reaction of the bisoprolol in the pressure-sensitive adhesive layer is remarkably inhibited, and there is hardly any decrease in bisoprolol attributable to this hydrolysis observed. Consequently, according to such a patch-containing packaging pouch, the effects of bisoprolol can be obtained that are essentially the same as those at the time of production even if the patch is stored for a long period of time.

A desiccant is preferably additionally housed within the packaging pouch of the patch-containing packaging pouch. The inside of the packaging pouch is preferably maintained at a value of the above-mentioned relative humidity as a result of this desiccant absorbing moisture. As a result, extraordinary procedures for drying the inside of the packaging pouch are not required, thereby facilitating maintenance of the relative humidity inside the packaging pouch at the above-mentioned value.

In this case, even if the pressure-sensitive adhesive layer of the patch contains a certain degree of moisture prior to packaging, since the patch is introduced into the packaging pouch together with a desiccant, the moisture in the pressure-sensitive adhesive layer is absorbed by the desiccant, thereby enabling the moisture content of the pressure-sensitive adhesive layer to be within a preferable range. In other words, the use of a desiccant makes it possible to maintain the relative humidity within the packaging pouch at the above-mentioned value, while simultaneously allowing the omission of drying procedures for the package material and so forth which were normally required during patch production.

The above-mentioned desiccant is preferably a desiccant comprised of a substance that physically adsorbs water, or in other words, a physical desiccant. Such desiccants are able to effectively adsorb moisture within the packaging pouch, making it possible to easily maintain the inside of the packaging pouch at the above-mentioned value of relative humidity.

In addition, the desiccant is preferably formed of a porous substance. More specifically, the desiccant is preferably formed of a porous substance formed of at least one type of material selected from the group comprising metal oxides, zeolite and clay minerals.

At least one type of compound selected from the group comprising styrene-isoprene-styrene block copolymer, polyisobutylene and acrylic polymer is preferable for the above-mentioned pressure-sensitive adhesive. These pressure-sensitive adhesives have satisfactory adhesion to skin and cause little irritation of the skin. In addition, according to a pressure-sensitive adhesive layer containing these pressure-sensitive adhesives and bisoprolol, the bisoprolol easily migrates from the pressure-sensitive adhesive layer to the skin, and as a result, the efficacy of the patch is demonstrated more effectively.

In addition, the packaging pouch of the patch-containing packaging pouch of the present invention preferably has at least a blocking layer that blocks the penetration of moisture. As a result, entrance of moisture from outside the packaging pouch is extremely low, thereby facilitating maintenance of the relative humidity therein at the above-mentioned value.

In addition, the packaging pouch preferably has a layer composed of polyacrylonitrile on the innermost side of said thereof. A layer composed of polyacrylonitrile has the property of impairing migration of bisoprolol from the pressure-sensitive adhesive layer of the patch. Consequently, decreases in stability based on migration of the bisoprolol to the packaging pouch can be inhibited by providing a layer composed of polyacrylonitrile on the innermost side of the packaging pouch.

### BRIEF DESCRIPTION OF THE DRAWINGS

FIG. 1 is a cross-sectional view of a preferable embodiment of a patch-containing packaging pouch of the present invention.

### BEST MODE FOR CARRYING OUT THE INVENTION

The following provides a detailed explanation of embodiments of the present invention with reference to the drawings.

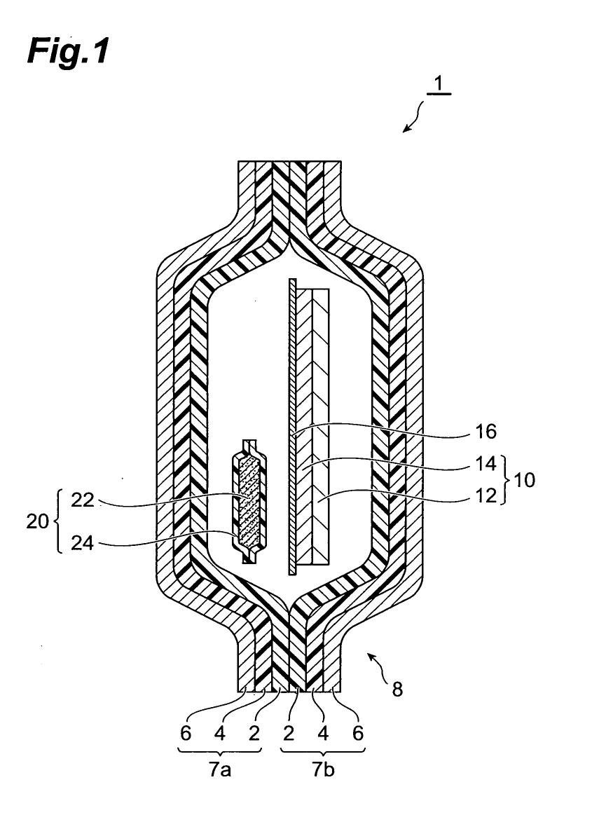

FIG. 1 is a cross-sectional view of a preferable embodiment of the patch-containing packaging pouch of the present invention. A patch-containing packaging pouch 1 has a packaging pouch 8 comprised of a pair of laminated package members 7a and 7b arranged in mutual opposition, and a patch 10 housed in the space within this packaging pouch 8. In addition, in the patch-containing packaging pouch 1 shown in FIG. 1, a packaged desiccant 20 is additionally housed within packaging pouch 8. In a patch-containing packaging pouch 1 having such a constitution, the relative humidity at 25° C. within packaging pouch 8 is maintained at 25% or less as will be described later.

Patch 10 is provided with a roughly rectangular support 12, and a pressure-sensitive adhesive layer 14 laminated roughly over the entire surface of one side of this support 12. In addition, a backing film 16 that can be separated from pressure-sensitive adhesive layer 14 is adhered to pressure-sensitive adhesive layer 14 in this patch 10.

There are no particular limitations on support 12 provided it is able to support pressure-sensitive adhesive layer 14, and it preferably has suitable flexibility from the viewpoint of enhancing adhesion of patch 10 to the skin. Examples of preferable constituent materials of support 12 include films composed of resin materials such as polymers in the manner of polyester, polypropylene, polyethylene, vinyl acetate and vinyl chloride, or polymers composed by polymerizing constituent monomers thereof (for example, ethylene-vinyl acetate copolymer), fabrics such as woven fabrics or non-woven fabrics formed from fibers composed of these resins, and compounds composed of these films and fabrics.

Pressure-sensitive adhesive layer 14 is composed of a pressure-sensitive adhesive composition containing a pressure-sensitive adhesive and a drug in the form of bisoprolol or pharmaceutically acceptable salt thereof (to be collectively referred to as a “bisoprolol compound”). The pressure-sensitive adhesive in the pressure-sensitive adhesive composition is preferably safe for the skin and has tackiness that enables the patch to be fixed on a skin surface at ordinary temperatures, and that which is composed of a material that is typically known to be a pressure-sensitive adhesive of a patch can be applied.

More specifically, an example of a pressure-sensitive adhesive is that which is composed of a base, a tackifier and a softening agent. Examples of bases in the pressure-sensitive adhesive of this constitution include natural rubber materials, synthetic rubber materials, acrylic adhesive materials and silicon adhesive materials. In particular, synthetic rubber materials and/or acrylic adhesive materials are preferable because of their superior tackiness and superior ability to release a drug. Examples of synthetic rubber materials include homopolymers such as polyisobutylene and polyisoprene, and copolymers in which they are contained. Styrene-isoprene-styrene block copolymer (SIS) and polyisobutylene are particularly preferable.

Examples of acrylic resin materials include homopolymers of (meth)acrylic acid (esters) such as acrylic acid, 2-ethylhexyl acrylate, methyl acrylate, butyl acrylate, hydroxyethyl acrylate and 2-ethylhexyl methacrylate, and copolymers containing at least one type of these (meth)acrylic acid (esters). Examples of such acrylic resin materials include 2-ethylhexyl acrylate-vinyl acetate copolymer, 2-ethylhexyl acrylate-vinyl acetate-acrylic acid copolymer, 2-ethylhexyl acrylate-vinyl acetate-hydroxyethyl acrylate copolymer, 2-ethylhexyl acrylate-vinyl acetate-hydroxyethyl acrylate-acrylic acid copolymer and 2-ethylhexyl acrylate-2-ethylhexyl methacrylate-dodecyl methacrylate copolymer. 2-ethylhexyl acrylate-vinyl acetate copolymer and 2-ethylhexyl acrylate-vinyl acetate-acrylic acid copolymer are particularly preferable.

In addition, the pressure-sensitive adhesive is preferably also a compound material combining a plurality of types of the above-mentioned adhesive materials. Examples of such compound materials include compound materials of SIS and acrylic adhesive, and more specifically, a compound material consisting of a mixture of SIS and 2-ethylhexyl acrylate-vinyl acetate-acrylic acid copolymer is preferable.

In addition, examples of the tackifier contained in the pressure-sensitive adhesive include alicyclic saturated hydrocarbon resins, rosin derivatives (such as rosin, glycerin esters of rosin, hydrogenated rosin, glycerin esters of hydrogenated rosin and pentaerythritol esters of rosin), terpene resins, petroleum resins and maleic acid resins. Alicyclic saturated hydrocarbon resins and hydrogenated rosin esters are particularly preferable. These tackifiers may be used alone or two or more types may be used in combination.

Moreover, examples of softening agents include petroleum-based oils (such as paraffin processed oils, naphthene processed oils and aromatic processed oils), squalane, squalene, vegetable oils (such as almond oil, olive oil, camellia oil, castor oil, tall oil and peanut oil), olefinic acid, silicon oil, dibasic acid esters (such as dibutyl phthalate and dioctyl phthalate), liquid rubber (such as polybutene and liquid isoprene rubber), liquid fatty acid esters (such as isopropyl myristate, hexyl laurate, diethyl sebacate and isopropyl sebacate), diethylene glycol, polyethylene glycol, glycol salicylate, propylene glycol, dipropylene glycol, triacetin, triethyl citrate and crotamiton. Among these, liquid paraffin, isopropyl myristate or diethyl sebacate is particularly preferable since it is able to impart suitable adhesion to skin. These softening agents may be used alone or two or more types may be used in combination.

Moreover, an example of a bisoprolol compound serving as a drug in the pressure-sensitive adhesive composition is bisoprolol hemifumarate which has an action of selectively blocking β₁-receptors.

In the pressure-sensitive adhesive composition, the content of a bisoprolol compound is preferably 1 to 50% by mass and more preferably 5 to 20% by mass. If the content of the bisoprolol compound is less than 1% by mass, it becomes difficult for the bisoprolol compound to be released from pressure-sensitive adhesive layer 14, and it tends to be difficult to administer an effective amount of drug at the time of use. On the other hand, if the content of the bisoprolol compound exceeds 50% by mass, the bisoprolol tends to be retained in pressure-sensitive adhesive layer 14, and the tackiness properties of pressure-sensitive adhesive layer 14 tend to decrease.

In addition, in the case the pressure-sensitive adhesive contains a base, a tackifier and a softening agent as previously described, the content of each component is preferably within the following ranges. Namely, within the total amount of the pressure-sensitive adhesive, the content of the base is preferably 10 to 90% by mass, the content of the tackifier is preferably 0 to 60% by mass, and the content of the softening agent is preferably 0 to 30% by mass.

There are no particular limitations on backing film 16 provided it can be separated from pressure-sensitive adhesive layer 14, and examples include a resin film such as polyethylene terephthalate (abbreviated as PET) or polyester which has been subjected to separation treatment (and preferably silicon treatment), or paper. Furthermore, this backing film 16 preferably has suitable rigidity so that wrinkles, twisting or torsion do not occur in patch 10. In addition, as shown in FIG. 1, backing film 16 preferably has a larger surface area than pressure-sensitive adhesive layer 14 to facilitate separation of backing film 16 during use of patch 10. In this case, said film 16 can be separated easily by grabbing the location of backing film 16 that protrudes from pressure-sensitive adhesive layer 14.

The size of patch 10 having this type of constitution can be suitably altered in consideration of the site where patch 10 is used, the drug dosage and so on. For example, patch 10 can be made to have a size that is normally used for a patch, and that having a surface area of 1 to 100 cm², and preferably 5 to 40 cm², can be applied. In addition, various values can be adopted for the thickness of each layer from the above-mentioned viewpoint, and the thickness of pressure-sensitive adhesive layer 14, for example, is 10 to 300 μm and preferably 25 to 150 μm.

Packaging pouch 8 for packaging the above-mentioned patch 10 is composed of a pair of roughly rectangular laminated package members 7a and 7b arranged in mutual opposition. Laminated package members 7a and 7b are film-like laminates having a roughly rectangular shape, and have a constitution in which a PAN layer 2 composed of polyacrylonitrile (PAN), an Al layer 4 composed of aluminum foil, and a PET layer 6 composed of PET, are laminated in that order starting from the inside. In addition, mutually opposed laminated package members 7a and 7b are joined at the outer edges thereof, and as a result, are sealed around their entire circumference. Furthermore, joining of the outer edges of each laminated member 7 can be carried out by heating sealing or using an adhesive.

Among each of the layers which compose laminated package members 7a and 7b, since PAN layer 2 is hardly any reactivity with the bisoprolol contained in pressure-sensitive adhesive layer 14 in patch 10 or absorption thereof, PAN layer 2 fulfills the role of preventing migration of drug from patch 10 to the packaging pouch.

Since Al layer 4 functions as a blocking layer that blocks penetration of moisture while also having superior properties that block the penetration of gases and light, it fulfills the role of keeping the inside of packaging pouch 8 airtight. In addition, since PET layer 6 has low oxygen permeability, it has the effect of further enhancing the airtightness of packaging pouch 8. According to a packaging pouch 8 provided with each of these layers, since migration of the bisoprolol compound from the patch can be inhibited, and the effects of the external atmosphere can be eliminated as much as possible, patch 10 can be stored for a long period of time.

In addition, the relative humidity at 25° C. within packaging pouch 8 is maintained at 25% or less in the patch-containing packaging pouch 1 of the present embodiment as previously described.

Here, since the drug in the form of a bisoprolol compound contained in pressure-sensitive adhesive layer 14 of patch 10 has the property of being hydrolyzed extremely easily, it was conventionally extremely difficult to apply a bisoprolol compound to a patch.

In contrast, according to the novel findings of the inventors of the present invention, the hydrolysis reaction of a bisoprolol compound, and particularly a bisoprolol compound contained in a pressure-sensitive adhesive layer in a patch, was determined to be inhibited considerably at the above-mentioned value of relative humidity (25% or less at 25° C.).

Since the relative humidity within packaging pouch 8 is maintained at 25% or less in patch-containing packaging pouch 1, there is extremely little occurrence of hydrolysis of the bisoprolol compound in pressure-sensitive adhesive layer 14 of patch 10 as previously described. Thus, according to patch-containing packaging pouch 1 of the present embodiment, the application of bisoprolol to a patch, although difficult in the past, becomes easy. In addition, the resulting patch 1 has extremely high storage stability in this aspect of patch-containing packaging pouch 1.

Furthermore, from the viewpoint of further enhancing the storage stability of patch 10 in patch-containing packaging pouch 1, the relative humidity at 25° C. in packaging pouch 8 is preferably 22% or less, more preferably 20% or less, even more preferably 17% or less, still even more preferably 15% or less and most preferably 10% or less. In addition, relative humidity is more preferably maintained at equal to or less than the above-mentioned values even at temperatures other than 25° C., such as at 40° C. or 60° C.

In consideration of the objective of preventing hydrolysis of the bisoprolol compound, it is ideal that moisture not be present in packaging pouch 8, namely that the relative humidity therein be 0%. However, in the case of generation of static electricity between patch 10 and packaging pouch 8 resulting in contact between the two, or when it is difficult to remove patch 10 from packaging pouch 8 for this reason, a slight amount of moisture may be present within packaging pouch 8 to avoid these problems. In this case, the value of relative humidity at 25° C. within packaging pouch 8 is preferably 2% or more.

As has been described above, in patch-containing packaging pouch 1 of the present embodiment, desiccant 20 is housed within packaging pouch 8 together with patch 10, and the above-mentioned value of relative humidity is achieved by this packaged desiccant 20. More specifically, as a result of packaged desiccant 20 housed within packaging pouch 8 absorbing (adsorbing) moisture present within packaging pouch 8, the inside of said packaging pouch 8 is dried and as a result, the relative humidity within packaging pouch 8 is within the numerical range described above.

This packaged desiccant 20 is composed of a desiccant packaging pouch 24 and a desiccant 22 housed within this packaging pouch 24. Desiccant packaging pouch 24 is able to house desiccant 22 therein, and can be applied without any particular limitations provided it does not obstruct drying by desiccant 22 and allows permeation of moisture. Examples of desiccant packaging pouch 24 include those composed of a moisture-permeable material such as a resin film composed of low-density polyethylene (LDPE), a fabric such as a woven fabric or a non-woven fabric or paper. In addition to these water-permeable materials, materials that are not permeable to water can also be used for the constituent material of desiccant packaging pouch 24. In the case of applying such a material, moisture permeability can be secured by providing holes, which allow penetration of moisture and of a size that does not expose desiccant 22. housed therein to the outside, in packaging pouch 24 composed of said material.

A desiccant composed of a known material which is typically used as a desiccant can be applied for desiccant 22 housed within this desiccant packaging pouch 24, an example of which is a desiccant composed of a substance having the ability to physically or chemically adsorb moisture. A physical desiccant which is physically able to adsorb moisture is particularly preferable for desiccant 22 because of its ease of handling and high drying capacity without significantly contaminating patch 10. A powdered substance composed of a porous substance is preferable for this type of physical desiccant.

More specifically, examples of porous substances include a porous substance composed of an amorphous porous substance in the form of a metal oxide, a porous substance composed of a crystalline porous substance in the form of zeolite, and a porous substance in the form of a crystalline or non-crystalline porous substance in the form of a clay mineral. Specific examples of metal oxide-based porous substances include silica gel and alumina, specific examples of zeolite-based porous substances include a molecular sieve, and specific examples of clay mineral-based porous substances include montmorillonite.

Silica gel, molecular sieve or montmorillonite is particularly preferable for the substance that composes desiccant 22. Furthermore, these may be used alone or two or more types may be used in combination. In the case of using a porous substance as described above for desiccant 22, there are no particular limitations on the size of the pores thereof, and a porous substance having mesopores or micropores and so on can be suitably selected and used.

Packaged desiccant 20 having this type of constitution is required to have performance that enables the relative humidity within packaging pouch 8 to attain the above-mentioned value. Namely, the amount of desiccant 22 retained by packaged desiccant 20 is the amount that said value of relative humidity to be maintained within packaging pouch 8 in patch-containing packaging pouch 1.

In addition, although the size of packaged desiccant 20 is also required to be suitably set based on a similar viewpoint, it is preferably as small as possible while still being able to achieve the above-mentioned relative humidity in order to reduce the overall size of patch-containing packaging pouch 1. More specifically, the size of packaged desiccant 20 is preferably of a size that is not larger than the surface area of patch 10 while also being as thin as possible.

Furthermore, in the patch-containing packaging pouch of the present invention, the value of relative humidity within packaging pouch 8 may also be achieved by another method in addition to the method using packaged desiccant 20 as described above.

For example, a method may be employed in which the moisture content adsorbed by pressure-sensitive adhesive layer 14 and so on is reduced as much as possible by carrying out a predetermined drying procedure, such as by placing patch 10 under conditions of heating and reduced pressure either during or after production of patch 10, and then sealing patch 10 in packaging pouch 8 capable of blocking external moisture immediately after said drying procedure. In addition, a method may also be employed in which dry nitrogen is sealed within packaging pouch 8 together with packaging patch 10. Furthermore, these methods can also be carried out in combination.

A patch-containing packaging pouch 1 having this type of constitution can be produced by a production method such as that described below.

During production of patch 10, after first mixing the constituent components of pressure-sensitive adhesive layer 14 consisting of a base, a tackifier, a softening agent and bisoprolol compound to form a pressure-sensitive adhesive composition, this composition is heated and melted (softened). Next, after coating the molten (softened) composition onto the surface of either a support 12 or backing film 16, the coated composition layer is affixed to the another one of the support 12 or backing film 16. As a result, a laminated is obtained having a structure in which backing film 16 is adhered to patch 10 in which pressure-sensitive adhesive layer 14 is formed on support 12. In addition, an example of another method consists of coating a solution in which the above-mentioned composition has been dissolved in a solvent such as toluene, hexane, heptane or ethyl acetate onto either support 12 or backing film 16, and after drying off the solvent in the coating, affixing in the manner described above to obtain a laminate of the same structure as described above.

Furthermore, during production of patch 10, it is preferable to remove moisture adsorbed in pressure-sensitive adhesive layer 14 during the course of production, such as by placing patch 10 under conditions of heating and reduced pressure, after forming pressure-sensitive adhesive layer 14 or after adhering backing film 16. However, since patch 10 is packaged within packaging pouch 8 together with packaged desiccant 20 in the preferable embodiment of patch-containing packaging pouch 1, moisture within pressure-sensitive adhesive layer 14 is adequately adsorbed by packaged desiccant 20. Consequently, the moisture content in pressure-sensitive adhesive layer 14 can be reduced to a suitable level even after patch 10 has been packaged. Thus, an extraordinary drying procedure as described above need not be carried out in a production method of patch-containing packaging pouch 1 having packaged desiccant 20 within packaging pouch 8.

Packaged desiccant 20 can be produced by, for example, preparing a pair of roughly rectangular films to compose desiccant packaging pouch 24, and after arranging these films in opposition to each other so as to sandwich desiccant 22, joining the peripheries thereof by heat sealing or adhesion. In addition, three sides of the above-mentioned pair of roughly rectangular films may be joined in advance, and then the remaining side may be closed after inserting desiccant 22 through the opening.

After arranging patch 10 and packaged desiccant 20 produced in the manner described above between the above-mentioned pair of laminated package members 7a and 7b, the outer edges of these laminated package members 7a and 7b are joined by heat sealing or adhesion. In addition, after joining three sides of this pair of laminated package members 7a and 7b in advance, the remaining side may be closed after inserting patch 10 and packaged desiccant 20 through the opening. Patch-containing packaging pouch 1 can be obtained in this manner.

The actions and effects indicated below are obtained by patch-containing packaging pouch 1 having the above-mentioned constitution.

First, in patch-containing packaging pouch 1, the relative humidity at 25° C. within packaging pouch 8 is maintained at 25% or less. This value of relative humidity is a value that enables hydrolysis of the bisoprolol compound contained pressure-sensitive adhesive layer 14 in patch 10 to be effectively inhibited as previously described. Thus, in patch-containing packaging pouch 1, it is extremely difficult for hydrolysis of the bisoprolol compound to proceed in pressure-sensitive adhesive layer 14. As a result, patch 10 can be stored for a long period of time, and patch 10 has sufficiently effective efficacy even after being stored for a long period of time.

In addition, in a preferable embodiment of patch-containing packaging pouch 1, the above-mentioned relative humidity value can be achieved simply by having packaged desiccant 20 present with patch 10. Consequently, an extraordinary drying procedure for removing moisture within packaging pouch 8 is not required during production or packaging of patch 10. Thus, according to patch-containing packaging pouch 1, in addition to patch 10 being able to be stored for a long period of time, the production processes of patch 10 and patch-containing packaging pouch 1 can be simplified.

In this manner, according to the patch-containing packaging pouch of the present invention, a bisoprolol compound can be applied to a patch even though this was extremely difficult in the past due to the high susceptibility to hydrolysis thereof. In this patch-containing packaging pouch, a patch containing bisoprolol in a pressure-sensitive adhesive layer has high storage stability. Consequently, said patch demonstrates an extremely low decrease in efficacy over time during storage.

Although the above has provided an explanation of an embodiment of the patch-containing packaging pouch of the present invention, the present invention is not necessarily limited to the above-mentioned embodiment, and various variations are possible. For example, in patch-containing packaging pouch 1, although both patch 10 and packaging pouch 8 have a roughly rectangular shape, their shape is not particularly limited to said shape, but rather may have a shape such as that of a circle or polygon.

In addition, although the above-mentioned embodiment is described such that packaging pouch 8 is composed of laminated package members 7a and 7b having a trilaminar structure, if the packaging pouch in the patch-containing packaging pouch of the present invention has the ability to block moisture, the packaging pouch may have a single-layer, two-layer or four or more layer laminated structure provided it satisfies this condition.

### EXAMPLES

Although the following provides a more detailed explanation of the present invention through examples thereof, the present invention is not limited to these examples.

<Fabrication of Patch>

### Production Example 1

Bisoprolol and softening agents in the form of liquid paraffin and diethyl sebacate were placed in a container and stirred followed by mixing well. This mixture was then mixed with a solution, in which a base in the form of SIS and an acrylic polymer (Duro-tak 2194, National Starch & Chemical) and a tackifier in the form of an alicyclic saturated hydrocarbon resin (Arkon P-100, Arakawa Chemical Industries) were dissolved in toluene, to prepare a coating liquid.

Next, after coating this coating liquid onto a silicon-treated backing film composed of PET, the toluene was removed by evaporation to obtain a pressure-sensitive adhesive layer, after which a support composed of PET was additionally adhered to this pressure-sensitive adhesive layer to obtain a patch in which the pressure-sensitive adhesive layer on the support was covered with the backing film. The surface area on one side of the resulting patch was made to be 10 cm². Furthermore, this production example was prepared so that the blended amounts of each component were made to be the contents shown in Table 1 below for each of the components in the pressure-sensitive adhesive layer.

TABLE 1

| Component | Content (mass %) |
|---|---|
| Bisoprolol | 10.0 |
| SIS | 10.0 |
| Acrylic copolymer | 26.5 |
| Alicyclic saturated hydrocarbon resin | 40.5 |
| Liquid paraffin | 5.0 |
| Diethyl sebacate | 8.0 |
| Total | 100 |

### Production Example 2

A bisoprolol compound in the form of bisoprolol hemifumarate, softening agents in the form of liquid paraffin and diethyl sebacate, and anhydrous sodium acetate were placed in a mortar and mixed well. This mixture was then mixed with a solution, in which a base in the form of SIS and an acrylic polymer (Duro-tak 2194, National Starch & Chemical) and a tackifier in the form of an alicyclic saturated hydrocarbon resin (Arkon P-100, Arakawa Chemical Industries) were dissolved in toluene, to prepare a coating liquid. A patch was then obtained in the same manner as Production Example 1 using the resulting coating liquid. Furthermore, this production example was prepared so that the blended amounts of each component were made to be the contents shown in Table 2 below for each of the components in the pressure-sensitive adhesive layer.

TABLE 2

| Component | Content (wt %) |
|---|---|
| Bisoprolol hemifumarate | 12.5 |
| SIS | 10.0 |
| Acrylic copolymer | 24.0 |
| Alicyclic saturated hydrocarbon resin | 35.0 |
| Liquid paraffin | 5.0 |
| Diethyl sebacate | 8.0 |
| Anhydrous sodium acetate | 5.5 |
| Total | 100 |

<Preparation of Packaged Desiccant>

(Packaged Desiccant A)

A desiccant in the form of a silica-based desiccant (SORB-IT (registered trademark), Sud Chemie) was packaged with a moisture-permeable desiccant packaging pouch to obtain Packaged Desiccant A.

(Packaged Desiccant B)

A desiccant in the form of a molecular sieve (TRI-SORB (registered trademark), Sud Chemie) was packaged with a moisture-permeable desiccant packaging pouch to obtain Packaged Desiccant B.

(Packaged Desiccant C)

A desiccant in the form of a clay mineral-based desiccant (DESIPAK (registered trademark), Sud Chemie) was packaged with a moisture-permeable desiccant packaging pouch to obtain Packaged Desiccant C.

### Example 1

Two roughly square laminated package members, having a PAN layer composed of PAN, an Al layer composed of aluminum foil, and a PET layer composed of PET laminated in that order, were prepared followed by arranging the package members in opposition to each other with the PAN layer on the inside. The patch of Production Example 1 and Packaged Desiccant A were sandwiched between this pair of laminated package members, and the outer edges of the pair of laminated package members were joined by heat sealing to obtain a patch-containing packaging pouch. The total inner surface area of the resulting patch-containing packaging pouch was 134 cm². In addition, the relative humidity within the packaging pouch was made to be 10%.

Furthermore, the relative humidity inside the packaging pouch (10%) was adjusted according to the following method. Namely, after first storing a sample in which only a desiccant was housed in the package for 24 hours under conditions of constant temperature and humidity, the relative humidity within the packaging pouch was measured. The same conditions as the sample in the case of the relative humidity value being 10% were applied as the conditions under which the relative humidity in the packaging pouch becomes 10% in the present example. Furthermore, the relative humidity within the packaging pouch was adjusted in the same manner in the following tests.

### Example 2

A patch-containing packaging pouch was prepared using the same procedure and conditions as Example 1 with the exception of using Packaged Desiccant B instead of Packaged Desiccant A.

### Example 3

A patch-containing packaging pouch was prepared using the same procedure and conditions as Example 1 with the exception of using Packaged Desiccant C instead of Packaged Desiccant A.

### Comparative Example 1

A patch-containing packaging pouch was obtained in the same manner as Example 1 with the exception of using for the packaging pouch additionally provided with a moisture absorbent layer composed of low-density polyethylene containing a filler in the form of magnesium sulfate between the PAN layer and Al layer of the packaging pouch used in Example 1, and not using a desiccant. Furthermore, the moisture absorbent layer has the property of moisture being adsorbed by the magnesium sulfate contained in said layer.

Furthermore, as a result of measuring the relative humidity in the packaging pouch after storing only the packaging pouch used in this comparative example for 24 hours under the conditions used to determine the relative humidity in Example 1 (temperature: 25° C., relative humidity: 60%), the relative humidity was determined to be 30%. On the basis of this result, use of this packaging pouch only results in conditions in which the relative humidity within the packaging pouch is 30%.

### Example 4

A patch-containing packaging pouch was prepared using the same procedure and conditions as Example 1 with the exception of using the patch obtained in Production Example 2 instead of the patch obtained in Production Example 1.

### Example 5

A patch-containing packaging pouch was produced using the same procedure and conditions as Example 4 with the exception of using Packaged Desiccant B instead of Packaged Desiccant A.

### Example 6

A patch-containing packaging pouch was produced using the same procedure and conditions as Example 4 with the exception of using Packaged Desiccant C instead of Packaged Desiccant A.

### Comparative Example 2

A patch-containing packaging pouch was prepared using the same procedure and conditions as Comparative Example 1 with the exception of using the patch obtained in Production Example 2 instead of the patch obtained in Production Example 1.

### Example 7

A patch-containing packaging pouch was obtained in the same manner as Example 1 with the exception of making the relative humidity within the packaging pouch 20%.

[Evaluation of Stability of Patch-Containing Packaging Pouches]

Each of the patch-containing packaging pouches obtained in Examples 1 to 7 and Comparative Examples 1 and 2 was stored for 3 months in a constant-temperature, constant-humidity chamber at a temperature of 40° C. and relative humidity of 75%. In addition, the patch-containing packaging pouches obtained in Example 1 and Comparative Example 1 were stored for 12 months in a constant-temperature, constant-humidity chamber at a temperature of 25° C. and relative humidity of 60%. The patch was removed from the patch-containing packaging pouch at completion of each storage period, and the content of bisoprolol or bisoprolol hemifumarate (to be collectively referred to as the “bisoprolol compound”) in each patch after storage was measured. The residual percentage (%) of the bisoprolol compound in each patch after storage was then calculated by comparing the content of the bisoprolol compound in each patch after storage with the content of the bisoprolol compound in the patch before storage.

Furthermore, the content of the bisoprolol compound in each patch and the residual percentage of the bisoprolol compound in each patch-containing packaging pouch were measured and calculated as described below. Namely, the backing film adhered to each patch after storage was separated, and the resulting patch was placed in a 50 mL centrifuge tube. 10 mL of an extraction liquid in the form of tetrahydrofuran were placed in this centrifuge tube followed by shaking the tube for 1 hour. Next, after adding an internal standard substance (isopropyl 4-aminobenzoate/methanol solution) to the extract, the solution was diluted with 50 mL of methanol for use as the test sample. Each of the resulting test samples was analyzed by high-performance liquid chromatography. The content of the bisoprolol compound contained in each patch was calculated based on a calibration curve obtained by preliminarily analyzing a standard solution of a known concentration.

On the other hand, patches identical to those obtained in Production Examples 1 and 2 were prepared for use a pre-storage patches, and the content of the bisoprolol compound contained in each patch was calculated in the same manner as described above using these patches.

The value of the content of the bisoprolol compound contained in the patch in each patch-containing packaging pouch after storage (Nᵢ), and the value of the content of the bisoprolol compound contained in the respectively corresponding patch before storage (N₀) were substituted into the relational expression shown in Equation (1) below, and the resulting value (Rᵢ) was taken to be the residual percentage (%) of the bisoprolol compound after storage in each patch-containing packaging pouch.
Rᵢ(%)=Nᵢ/N₀×100 (1)

The residual percentages of the bisoprolol compound in the case of storing each patch-containing packaging pouch of Examples 1 to 7 and Comparative Examples 1 and 2 for 3 months in a constant-temperature, constant-humidity chamber at a temperature of 40° C. and relative humidity of 75% are shown in Table 3 together with the values of relative humidity in the packaging pouch for each patch-containing packaging pouch. In addition, the residual percentages of the bisoprolol compound in the case of storing the patch-containing packaging pouches of Example 1 and Comparative Example 1 for 12 months in a constant-temperature, constant-humidity chamber at a temperature of 25° C. and relative humidity of 60% are shown in Table 4 together with the values of relative humidity in the packaging pouch for each patch-containing packaging pouch.

TABLE 3

| Patch contained in | | Relative |
|---|---|---|
| packaging pouch | Rᵢ (%) | humidity (%) |
| Example 1 | 100 | 10 |
| Example 2 | 98.7 | 10 |
| Example 3 | 99.1 | 10 |
| Comparative Example 1 | 96.9 | 30 |
| Example 4 | 100 | 10 |
| Example 5 | 98.5 | 10 |
| Example 6 | 98.3 | 10 |
| Comparative Example 2 | 95.7 | 30 |
| Example 7 | 99.3 | 20 |

TABLE 4

| Patch contained in | | |
|---|---|---|
| packaging pouch | Rᵢ (%) | Relative humidity (%) |
| Example 1 | 100 | 10 |
| Comparative Example 1 | 96.8 | 30 |

According to Tables 3 and 4, the patch-containing packaging pouches of the present invention (Examples 1 to 7), in which the relative humidity within the packaging pouch was 25% or less as a result of inserting a desiccant into the packaging pouch, demonstrated higher residual percentages of the bisoprolol compound in the patch as compared with the patch-containing packaging pouches of Comparative Examples 1 and 2, in which the relative humidity was higher than 25%, under either storage conditions. As a result, the patch-containing packaging pouch of the present invention was determined to allow long-term storage of a patch containing a bisoprolol compound.

As described above, according to the patch-containing packaging pouch of the present invention, hydrolysis reactions of bisoprolol in a patch whose pressure-sensitive adhesive layer contains bisoprolol can be inhibited, thereby enabling the patch to be stably stored for a long period of time.

1. A patch-containing packaging pouch comprising: a packaging pouch; and a patch, housed within the packaging pouch, in which a pressure-sensitive adhesive layer is formed on one side of a support, wherein
the pressure-sensitive adhesive layer is formed of a pressure-sensitive adhesive composition containing a pressure-sensitive adhesive and bisoprolol or a pharmaceutically acceptable salt thereof, and
relative humidity inside the packaging pouch at 25° C. is maintained at 25% or less.

2. The patch-containing packaging pouch according to claim 1, wherein the relative humidity is maintained at 10% or less.

3. The patch-containing packaging pouch according to claim 1, wherein a desiccant is further housed within the packaging pouch.

4. The patch-containing packaging pouch according to claim 3, wherein the desiccant is a desiccant formed of a substance which physically adsorbs moisture.

5. The patch-containing packaging pouch according to claim 3, wherein the desiccant is a desiccant formed of a porous substance.

6. The patch-containing packaging pouch according to claim 3, wherein the desiccant is a desiccant formed of a porous substance formed of at least one type of material selected from the group comprising a metal oxide, zeolite and a clay mineral.

7. The patch-containing packaging pouch according to claim 1, wherein the pressure-sensitive adhesive contains at least one type of compound selected from the group comprising a styrene-isoprene-styrene block copolymer, polyisobutylene and an acrylic polymer.

8. The patch-containing packaging pouch according to claim 1, wherein the packaging pouch has a blocking layer that blocks penetration of moisture.

9. The patch-containing packaging pouch according to claim 1, wherein the packaging pouch has layer formed from polyacrylonitrile on the innermost side of said packaging pouch.
